(19) United States
(12) Patent Application Publication
(10) Pub. No.: US 2010/0105875 A1
(43) Pub. Date: Apr. 29, 2010

(54) Antibodies against neisserial factor H binding protein

(76) Inventors: Maria Scarselli, Siena (IT); Mariagrazia Pizza, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/448,126

(22) Filed: Jun. 8, 2009

Related U.S. Application Data
(60) Provisional application No. 61/131,597, filed on Jun. 9, 2008.

(51) Int. Cl. C07K 16/00

(52) U.S. Cl. 530/387.9; 530/387.1

(57) ABSTRACT

Monoclonal antibody MAb502 binds to an epitope of meningococcal fHbp protein including Arg-204. Related antibodies are provided.

This application claims priority from U.S. provisional application 61/131,597 (filed 9 Jun. 2008), the complete contents of which are incorporated herein by reference.

### TECHNICAL FIELD

This invention relates to the Neisserial factor H binding protein (fHbp).

### BACKGROUND ART

Neisseria meningitidis is a Gram-negative encapsulated bacterium which colonises the upper respiratory tract of approximately 10% of human population. Although polysaccharide and conjugate vaccines are available against serogroups A, C, W135 and Y, this approach cannot be applied to serogroup B because the capsular polysaccharide is a polymer of polysialic acid, which is a self antigen in humans. To develop a vaccine against serogroup B, surface-exposed proteins contained in outer membrane vesicles (OMVs) have been used. These vaccines elicit serum bactericidal antibody responses and protect against disease, but they fail to induce cross-strain protection [1]. Some workers are therefore focusing on specific meningococcal antigens for use in vaccines [2].

One such antigen is the meningococcal factor H binding protein (fHbp), also known as protein ‘741’[SEQ IDs 2535 & 2536 in ref. 3], ‘NMB1870’, ‘GNA1870’ [refs. 4-6, following ref. 2], ‘P2086’, ‘LP2086’ or ‘ORF2086’ [7-9]. This lipoprotein is expressed across all meningococcal serogroups and has been found in multiple meningococcal strains. fHbp sequences have been grouped into three families [4] (referred to herein as families I, II & Ill), and it has been found that serum raised against a given family is bactericidal within the same family, but is not active against strains which express one of the other two families i.e. there is intra-family cross-protection, but not inter-family cross-protection.

Full-length mature fHbp has the following amino acid sequence (SEQ ID NO: 1) in strain MC58:

CSSGGGGVAADIGAGLADALTAPLDHKDKGLQSLTLDQSVRKNEKLKLAA
QGAEKTYGNGDSLNTGKLKNDKVSRFDFIRQIEVDGQLITLESGEFQVYK
QSHSALTAFQTEQTQDSEHSGKMVAKRQFRIGDIAGEHTSFDKLPEGGRA
TYRGTAFGSDDAGGKLTYTTDFAAKQGNGKIEHLKSPELNVDLAAADIKP
DGKRHAVISGSVLYNQAEKGSYSLGIFGGKAQEVAGSAEVKTVNGTRHIG
LAAKQ

The structure of fHbp's C-terminal immunodominant domain (‘fHbpC’) has been determined by NMR [10]. This part of the protein forms an eight-stranded β-barrel, whose strands are connected by loops of variable lengths. The barrel is preceded by a short α-helix and by a flexible N-terminal tail.

Reference 11 discloses six monoclonal anti-fHbp antibodies (JAR1 to JAR6). Reference 12 further discloses JAR10, JAR11 and JAR13.

It is an object of the invention to provide further and improved antibodies that bind to fHbp.

### DISCLOSURE OF THE INVENTION

The inventors have studied a monoclonal anti-fHbp antibody referred to as ‘MAb502’ [13]. This antibody is bactericidal and recognizes a conformational epitope within a well-defined area of the immunodominant C-terminal domain of fHbp. This epitope includes amino acids Gly-148, Arg-149 and Arg-204 of the MC58 fHbp sequence (SEQ ID NO: 1).

In one embodiment the invention provides an antibody having (i) a heavy chain comprising amino acid sequence SEQ ID NO: 2 and/or (ii) a light chain comprising amino acid sequence SEQ ID NO: 3.

In another embodiment, the invention provides an antibody that binds to an epitope of MC58 fHbp, wherein the epitope comprises amino acids Gly-148, Arg-149 and Arg-204.

In another embodiment, the invention provides an antibody that binds to an epitope of MC58 fHbp, wherein the epitope comprises amino acids Pro-145, Gly-147, Gly-148 and Arg-204.

In another embodiment, the invention provides an antibody having one or more (e.g. 1, 2 or 3) CDRs from within SEQ ID NO: 2. These CDRs may comprise SEQ ID NO: 4, 5 and/or 6.

In another embodiment, the invention provides an antibody having one or more (e.g. 1, 2 or 3) CDRs from within SEQ ID NO: 3. These CDRs may comprise SEQ ID NO: 7, 8 and/or 9.

The inventor also provides antibodies that bind to the same epitope as an antibody having (i) a heavy chain comprising amino acid sequence SEQ ID NO: 2 and (ii) a light chain comprising amino acid sequence SEQ ID NO: 3.

The inventor also provides antibodies that compete for binding to fHbp with an antibody having (i) a heavy chain comprising amino acid sequence SEQ ID NO: 2 and (ii) a light chain comprising amino acid sequence SEQ ID NO: 3.

### Antibodies

Antibodies of the invention may take various forms, but preferred antibodies are human antibodies. Unlike non-human antibodies, human antibodies will not elicit an immune response directed against their constant domains when administered to humans, although they may sometimes elicit human anti-idiotypic antibodies. Moreover, their variable domains are 100% human (in particular the framework regions of the variable domains are 100% human, in addition to the complementarity determining regions [CDRs]) and so will not elicit an immune response directed against the variable domain framework regions when administered to humans. Thus human antibodies do not include any sequences that do not have a human origin.

Human antibodies can be prepared by various means. For example, human B cells producing an antigen of interest can be immortalized e.g. by infection with Epstein Barr Virus (EBV). A preferred method for producing human monoclonal antibodies is disclosed in references 14 & 15, in which a human B memory lymphocyte specific for a target antigen is transformed using EBV in the presence of a polyclonal B cell activator. Human monoclonal antibodies can also be produced in non-human hosts by replacing the host's own immune system with a functioning human immune system e.g. into Scid mice or Trimera mice. Mice transgenic for human Ig loci have been successfully used for generating human monoclonal antibodies e.g. the “xeno-mouse” from Abgenix [16]. Phage display has also been successful for generating human antibodies [17], and led to the Humira™ product.

Rather than use human antibodies, the CDR sequences from a non-human antibody can be transferred into a human variable domain in order to create further antibodies sharing their antigen-binding specificity, in the process known as ‘CDR grafting’ [18-23]. The H1, H2 and H3 CDRs may be transferred together into an acceptor V_{H} domain, but it may also be adequate to transfer only one or two of them [21]. Similarly, one two or all three of the L1, L2 and L3 CDRs may be transferred into an acceptor V_{L} domain. Preferred antibodies will have 1, 2, 3, 4, 5 or all 6 of the donor CDRs. Where only one CDR is transferred, it will typically not be the L2 CDR, which is usually the shortest of the six. Typically the donor CDRs will all be from the same antibody, but it is also possible to mix them e.g. to transfer the light chain CDRs from a first antibody and the heavy chain CDRs from a second antibody.

By Kabat numbering [24], the CDRs in a light chain variable region are amino acids 24-34 (L1), 50-56 (L2) & 89-97 (L3), and the CDRs in a heavy chain variable region are amino acids 31-35 (H1), 50-65 (H2) and 95-102 (1-13). By Chothia numbering [25], the CDRs in a light chain variable region are amino acids 26-32 (L1), 50-52 (L2) & 91-96 (L3), and the CDRs in a heavy chain variable region are amino acids 26-32 (H1), 53-55 (H2) and 96-101 (H3). Framework residues are variable domain residues other than the CDRs.

As an alternative to CDR grafting, the process of ‘SDR grafting’ may be used [26,27], in which only the specificity-determining residues from within the CDRs are transferred.

The transfer of CDRs or SDRs from a donor variable domain into an acceptor domain may be accompanied by the modification of one or more framework residues, to give a humanised antibody.

Antibodies of the invention may be native antibodies, as naturally found in mammals. Native antibodies are made up of heavy chains and light chains. The heavy and light chains are both divided into variable domains and constant domains. The ability of different antibodies to recognize different antigens arises from differences in their variable domains, in both the light and heavy chains. Light chains of native antibodies in vertebrate species are either kappa (κ) or lambda (λ), based on the amino acid sequences of their constant domains. The constant domain of a native antibody's heavy chains will be α, δ, ε, γ or μ, giving rise respectively to antibodies of IgA, IgD, IgE, IgG, or IgM class. Classes may be further divided into subclasses or isotypes e.g. IgG1, IgG2, IgG3, IgG4, IgA, IgA2, etc. Antibodies may also be classified by allotype e.g. a γ heavy chain may have G1m allotype a, f, x or z, G2m allotype n, or G3m allotype b0, b1, b3, b4, b5, c3, c5, g1, g5, s, t, u, or v; a κ light chain may have a Km(1), Km(2) or Km(3) allotype. A native IgG antibody has two identical light chains (one constant domain C_{L} and one variable domain V_{L}) and two identical heavy chains (three constant domains C_{H}1 C_{H}2 & C_{H}3 and one variable domain V_{H}), held together by disulfide bridges. The domain and 3D structures of the different classes of native antibodies are well known.

Where an antibody of the invention has a light chain with a constant domain, it may be a κ or λ light chain. Where an antibody of the invention has a heavy chain with a constant domain, it may be a α, δ, ε, γ or μ heavy chain. Heavy chains in the γ class (i.e. IgG antibodies) are preferred. The IgG1 subclass is preferred. The Synagis™ antibody is IgG1 with a κ light chain. Antibodies of the invention may have any suitable allotype (see above).

Antibodies of the invention may be fragments of native antibodies that retain antigen binding activity. For instance, papain digestion of native antibodies produces two identical antigen-binding fragments, called “Fab” fragments, each with a single antigen-binding site, and a residual “Fc” fragment without antigen-binding activity. Pepsin treatment yields a “F(ab′)₂” fragment that has two antigen-binding sites. “Fv” is the minimum fragment of a native antibody that contains a complete antigen-binding site, consisting of a dimer of one heavy chain and one light chain variable domain. Thus an antibody of the invention may be Fab, Fab′, F(ab′)₂, Fv, or any other type, of fragment of a native antibody.

An antibody of the invention may be a “single-chain Fv” (“scFv” or “sFv”), comprising a V_{H} and V_{L} domain as a single polypeptide chain [28-30]. Typically the V_{H} and V_{L} domains are joined by a short polypeptide linker (e.g. >12 amino acids) between the V_{H} and V_{L} domains that enables the scFv to form the desired structure for antigen binding. A typical way of expressing scFv proteins, at least for initial selection, is in the context of a phage display library or other combinatorial library [31-33]. Multiple scFvs can be linked in a single polypeptide chain [34].

An antibody of the invention may be a “diabody” or “triabody” etc. [35-38], comprising multiple linked Fv (scFv) fragments. By using a linker between the V_{H} and V_{L} domains that is too short to allow them to pair with each other (e.g. <12 amino acids), they are forced instead to pair with the complementary domains of another Fv fragment and thus create two antigen-binding sites.

An antibody of the invention may be a single variable domain or VHH antibody. Antibodies naturally found in camelids (e.g. camels and llamas) and in sharks contain a heavy chain but no light chain. Thus antigen recognition is determined by a single variable domain, unlike a mammalian native antibody [39-41]. The constant domain of such antibodies can be omitted while retaining antigen-binding activity. One way of expressing single variable domain antibodies, at least for initial selection, is in the context of a phage display library or other combinatorial library [42].

An antibody of the invention may be a “domain antibody” (dAb). Such dAbs are based on the variable domains of either a heavy or light chain of a human antibody and have a molecular weight of approximately 13 kDa (less than one-tenth the size, of a full antibody). By pairing heavy and light chain dAbs that recognize different targets, antibodies with dual specificity can be made. dAbs are cleared from the body quickly, but can be sustained in circulation by fusion to a second dAb that binds to a blood protein (e.g. to serum albumin), by conjugation to polymers (e.g. to a polyethylene glycol), or by other techniques.

An antibody of the invention may be a variable lymphocyte receptor, as naturally found in lamprey and hagfish. References 43 and 44 disclose the production of such monoclonal antibodies against antigens of interest.

As mentioned above, an antibody of the invention may be a CDR-grafted antibody.

An antibody of the invention may be a chimeric antibody, having constant domains from one organism (e.g. a human) but variable domains from a different organism (e.g. non-human). Chimerisation of antibodies was originally developed in order to facilitate the transfer of antigen specificity from easily-obtained murine monoclonal antibodies into a human antibody, thus avoiding the difficulties of directly generating human monoclonal antibodies.

Thus the term “antibody” as used herein encompasses a range of proteins having diverse structural features (usually including at least one immunoglobulin domain having an all-β protein fold with a 2-layer sandwich of anti-parallel β-strands arranged in two β-sheets), but all of the proteins possess the ability to bind to fHbp.

Antibodies of the invention may include a single antigen-binding site (e.g. as in a Fab fragment or a scFv) or multiple antigen-binding sites (e.g. as in a F(ab′)₂ fragment or a diabody or a native antibody). Where an antibody has more than one antigen-binding site then advantageously it can result in cross-linking of antigens.

Where an antibody has more than one antigen-binding site, the antibody may be mono-specific (i.e. all antigen-binding sites recognize the same antigen) or it may be multi-specific (i.e. the antigen-binding sites recognise more than one antigen). Thus, in a multi-specific antibody, at least one antigen-binding site will recognise a pathogen factor and at least one antigen-binding site will recognise a different antigen.

An antibody of the invention may include a non-protein substance e.g. via covalent conjugation. For example, an antibody may include a radio-isotope e.g. the Zevalin™ and Bexxar™ products include ⁹⁰Y and ¹³¹I isotopes, respectively. As a further example, an antibody may include a cytotoxic molecule e.g. Mylotarg™ is linked to N-acetyl-γ-calicheamicin, a bacterial toxin. As a further example, an antibody may include a covalently-attached polymer, e.g. attachment of polyoxyethylated polyols or polyethylene glycol (PEG), has been reported to increase the circulating half-life of antibodies.

In some embodiments of the invention, an antibody can include one or more constant domains (e.g. including C_{H} or C_{L} domains). As mentioned above, the constant domains may form a κ or λ light chain or an α, δ, ε, γ or μ heavy chain. Where an antibody of the invention includes a constant domain, it may be a native constant domain or a modified constant domain. A heavy chain may include either three (as in α, γ, δ classes) or four (as in μ, ε classes) constant domains. Constant domains are not involved directly in the binding interaction between an antibody and an antigen, but they can provide various effector functions, including but not limited to: participation of the antibody in antibody-dependent cellular cytotoxicity (ADCC); C1q binding; complement dependent cytotoxicity; Fc receptor binding; phagocytosis; and down-regulation of cell surface receptors.

The constant domains can form a “Fc region”, which is the C-terminal region of a native antibody's heavy chain. Where an antibody of the invention includes a Fc region, it may be a native Fc region or a modified Fc region. A Fc region is important for some antibodies' functions e.g. the activity of Herceptin™ is Fc-dependent. Although the boundaries of the Fc region of a native antibody may vary, the human IgG heavy chain Fc region is usually defined to stretch from an amino acid residue at position Cys226 or Pro230 to the heavy chain's C-terminus. The Fc region will typically be able to bind one or more Fc receptors, such as a FcγRI (CD64), a FcγRII (e.g. FcγRIIA, FcγRIIB1, FcγRIIB2, FcγRIIC), a FcγRIII (e.g. FcγRIIIA, FcγRIIIB), a FcRn, FcaR (CD89), FcδR, FcμR, a FcεRI (e.g. FcεRIαβγ₂ or FcERIαγ₂), FcεRII (e.g. FcεRIIA or FcεRIIB), etc. The Fc region may also or alternatively be able to bind to a complement protein, such as C1q. Modifications to an antibody's Fc region can be used to change its effector function(s) e.g. to increase or decrease receptor binding affinity. For instance, reference 45 reports that effector functions may be modified by mutating Fc region residues 234, 235, 236, 237, 297, 318, 320 and/or 322. Similarly, reference 46 reports that effector functions of a human IgG1 can be improved by mutating Fc region residues (EU Index Kabat numbering) 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 and/or 439. Modification of Fc residues 322, 329 and/or 331 is reported in reference 47 for modifying C1q affinity of human IgG antibodies, and residues 270, 322, 326, 327, 329, 331, 333 and/or 334 are selected for modification in reference 48. Mapping of residues important for human IgG binding to FcRI, FcRII, FcRIII, and FcRn receptors is reported in reference 49, together with the design of variants with improved FcR-binding properties. Mutation of the Fc region of available monoclonal antibodies to vary their effector functions is known e.g. reference 50 reports mutation studies for RITUXAN™ to change C1q-binding, and reference 51 reports mutation studies for NUMAX™ to change FcR-binding, with mutation of residues 252, 254 and 256 giving a 10-fold increase in FcRn-binding without affecting antigen-binding.

Antibodies of the invention will typically be glycosylated. N-linked glycans attached to the C_{H}2 domain of a heavy chain, for instance, can influence C1q and FcR binding [49], with aglycosylated antibodies having lower affinity for these receptors. The glycan structure can also affect activity e.g. differences in complement-mediated cell death may be seen depending on the number of galactose sugars (0, 1 or 2) at the terminus of a glycan's biantennary chain. An antibody's glycans preferably do not lead to a human immunogenic response after administration.

Antibodies of the invention can be prepared in a form free from products with which they would naturally be associated. Contaminant components of an antibody's natural environment include materials such as enzymes, hormones, or other host cell proteins.

Antibodies of the invention can be used directly (e.g. as the active ingredient for pharmaceuticals or diagnostic reagents), or they can be used as the basis for further development work. For instance, an antibody may be subjected to sequence alterations or chemical modifications in order to improve a desired characteristic e.g. binding affinity or avidity, pharmacokinetic properties (such as in vivo half-life), etc. Techniques for modifying antibodies in this way are known in the art. For instance, an antibody may be subjected to “affinity maturation”, in which one or more residues (usually in a CDR) is mutated to improve its affinity for a target antigen. Random or directed mutagenesis can be used, but reference 52 describes affinity maturation by V_{H} and V_{L} domain shuffling as an alternative to random point mutation. Reference 53 reports how NUMAX™ was derived by a process of in vitro affinity maturation of the CDRs of the heavy and light chains of SYNAGIS™, giving an antibody with enhanced potency and 70-fold greater binding affinity for RSV F protein.

Preferred antibodies of the invention are specific for one of the pathogen factors described below. Thus the antibody will have a tighter binding affinity for that antigen than for an arbitrary control antigen e.g. than for a human protein or for a meningococcal PorA protein. Preferred antibodies have nanomolar or picomolar affinity constants for target antigens e.g. 10⁻⁹ M, 10⁻¹⁰ M, 10⁻¹¹ M, 10⁻¹² M, 10⁻¹³ M or tighter). Such affinities can be determined using conventional analytical techniques e.g. using surface plasmon resonance techniques as embodied in BlAcore™ instrumentation and operated according to the manufacturer's instructions.

The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that all have the same amino acid sequence and recognize the same epitope in the same target protein. Thus a monoclonal antibody may be produced using any suitable protein synthesis system, including immune cells, non-immune cells, acellular systems, etc. This usage is usual in the field: the product datasheets for the CDR-grafted humanised antibody Synagis™ expressed in a murine myeloma NSO cell line, the humanised antibody Herceptin™ expressed in a CHO cell line, and the phage-displayed antibody Humira™ expressed in a CHO cell line all refer the products as monoclonal antibodies.

### Nucleic Acids and Recombinant Antibody Expression

The invention also encompasses nucleic acid sequences encoding antibodies of the invention. Where an antibody of the invention has more than one chain (e.g. a heavy chain and a light chain), the invention encompasses nucleic acids encoding each chain. The invention also encompasses nucleic acid sequences encoding the amino acid sequences of CDRs of antibodies of the invention.

Nucleic acids encoding the antibodies can be prepared from cells, viruses or phages that express an antibody of interest. For instance, nucleic acid (e.g. mRNA transcripts, or DNA) can be prepared from an immortalised B cell of interest, and the gene(s) encoding the antibody of interest can then be cloned and used for subsequent recombinant expression. Expression from recombinant sources is more common for pharmaceutical purposes than expression from B cells or hybridomas e.g. for reasons of stability, reproducibility, culture ease, etc. Methods for obtaining and sequencing immunoglobulin genes from B cells are well known in the art e.g. see reference 54. Thus various steps of culturing, sub-culturing, cloning, sub-cloning, sequencing, nucleic acid preparation, etc. can be performed in order to perpetuate the antibody expressed by a cell or phage of interest. The invention encompasses all cells, nucleic acids, vectors, sequences, antibodies etc. used and prepared during such steps.

The invention provides a method for preparing one or more nucleic acid molecules (e.g. heavy and light chain genes) that encodes an antibody of interest, comprising the steps of: (i) providing an immortalised B cell clone expressing an antibody of interest; (ii) obtaining from the B cell clone nucleic acid that encodes the antibody of interest. The nucleic acid obtained in step (ii) may be inserted into a different cell type, or it may be sequenced.

The invention also provides a method for preparing a recombinant cell, comprising the steps of: (i) obtaining one or more nucleic acids (e.g. heavy and/or light chain genes) from a B cell clone that encodes an antibody of interest; and (ii) inserting the nucleic acid into an expression host in order to permit expression of the antibody of interest in that host.

Similarly, the invention provides a method for preparing a recombinant cell, comprising the steps of: (i) sequencing nucleic acid(s) from a B cell clone that encodes the antibody of interest; and (ii) using the sequence information from step (i) to prepare nucleic acid(s) for inserting into an expression host in order to permit expression of the antibody of interest in that host.

Recombinant cells produced in these ways can then be used for expression and culture purposes. They are particularly useful for expression of antibodies for large-scale pharmaceutical production.

The invention provides a method for preparing an antibody of the invention, comprising a step of culturing a cell such that it produces the antibody. The methods may further comprise a step of recovering the antibody that has been produced, to provide a purified antibody. A cell used in these methods may, as described elsewhere herein, be a recombinant cell, an immortalised B cell, or any other suitable cell. Purified antibody from these methods can then be used in pharmaceutical and/or diagnostic compositions, etc.

Cells for recombinant expression include bacteria, yeast and animal cells, particularly mammalian cells (e.g. CHO cells, human cells such as PER.C6 (ECACC deposit 96022940 [55]), NSO cells (ECACC deposit 85110503) or HKB-11 [56,57] cells), etc.), as well as plant cells. Preferred expression hosts can glycosylate the antibody of the invention, particularly with carbohydrate structures that are not themselves immunogenic in humans (see above). Expression hosts that can grow in serum-free media are preferred. Expression hosts that can grow in culture without the presence of animal-derived products are preferred.

The expression host may be cultured to give a cell line.

Nucleic acids used with the invention may be manipulated to insert, delete or amend certain nucleic acid sequences. Changes from such manipulation include, but are not limited to, changes to introduce restriction sites, to amend codon usage, to add or optimise transcription and/or translation regulatory sequences, etc. It is also possible to change the nucleic acid to alter the encoded amino acids. For example, it may be useful to introduce one or more (e.g. 1, 2, 3, 4, 5, 6, 7, 8, 9, 10, etc.) amino acid substitutions, one or more (e.g. 1, 2, 3, 4, 5, 6, 7, 8, 9, 10, etc.) amino acid deletions, and/or one or more (e.g. 1, 2, 3, 4, 5, 6, 7, 8, 9, 10, etc.) amino acid insertions into the antibody's amino acid sequence. Such point mutations can modify effector functions, antigen-binding affinity, post-translational modifications, immunogenicity, etc., can introduce amino acids for the attachment of covalent groups (e.g. labels) or can introduce tags (e.g. for purification purposes). Mutations can be introduced in specific sites or can be introduced randomly, followed by selection (e.g. molecular evolution).

Nucleic acids of the invention may be present in a vector (such as a plasmid) e.g. in a cloning vector or in an expression vector. Thus a sequence encoding an amino acid sequence of interest may be downstream of a promoter such that its transcription is suitable controlled. The invention provides such vectors, and also provides cells containing them.

The invention also provides an immortalised human B cell that can secrete an antibody of the invention.

### Antibody-Based Pharmaceutical Compositions

The use of antibodies as the active ingredient of pharmaceuticals is now widespread, including products such as Herceptin™ (trastuzumab) and Synagis™ (palivizumab). Synagis™ and Numax™ (motavizumab) in particular are effective in preventing pathogen-caused disease. The invention thus provides a pharmaceutical composition containing one or more antibody(ies) of the invention. Techniques for purification of monoclonal antibodies to a pharmaceutical grade are well known in the art.

A pharmaceutical composition will usually contain one or more pharmaceutically acceptable carriers and/or excipient(s). A thorough discussion of such components is available in reference 58. These may include liquids such as water, saline, glycerol and ethanol. Additionally, auxiliary substances, such as wetting or emulsifying agents or pH buffering substances, may be present in such compositions.

Pharmaceutical compositions may be prepared in various forms e.g. as injectables, either as liquid solutions or suspensions. Solid forms suitable for solution in, or suspension in, liquid vehicles prior to injection can also be prepared (e.g. a lyophilised composition, like Synagis™ and Herceptin™, for reconstitution with sterile water or buffer, optionally containing a preservative). The composition may be prepared for topical administration e.g. as an ointment, cream or powder. The composition may be prepared for oral administration e.g. as a tablet or capsule, as a spray, or as a syrup (optionally flavoured), in which case it will usually contain agents to protect the active ingredients from degradation. The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted (e.g. with sterile water or a sterile buffer) at the time of use, prior to administration to a patient e.g. an antibody can be provided in dry form.

Preferred pharmaceutical forms for administration of antibodies include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain carriers/excipients such as suspending, preservative, stabilising and/or dispersing agents.

Pharmaceutical compositions will generally have a pH between 5.5 and 8.5, preferably between 6 and 8, and more preferably about 7. The pH may be maintained by a buffer.

The composition will usually be sterile. The composition will usually be non-pyrogenic e.g. containing <1 EU (endotoxin unit, a standard measure) per dose, and preferably <0.1 EU per dose. The composition is preferably gluten free. The composition may be substantially isotonic with respect to humans.

Compositions may include an antimicrobial and/or preservative.

Compositions may comprise a detergent. Where present, detergents are generally used at low levels e.g. <0.01%.

Compositions may include sodium salts (e.g. sodium chloride) to give tonicity. A concentration of 10±2 mg/ml NaCl is typical.

Compositions may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material.

Compositions may include free amino acids e.g. histidine. For instance, reference 59 discloses an improved aqueous formulation for the Synagis™ antibody comprising histidine in an aqueous carrier.

Pharmaceutical compositions will include an effective amount of the active ingredient. The concentration of the ingredient in a composition will, of course, vary according to the volume of the composition to be delivered, and known antibody-based pharmaceuticals provide guidance in this respect. For example, Synagis™ is provided for reconstitution to give 50 mg antibody in 0.5 ml or 100 mg of antibody in 1.0 ml. The appropriate volume is delivered to a patient based on their recommended dose.

Once formulated, the compositions of the invention can be administered directly to the subject (see below). It is preferred that the compositions are adapted for administration to human subjects. This will generally be in liquid (e.g. aqueous) form.

In compositions that include antibodies, particularly pharmaceutical compositions, the antibodies preferably make up at least 50% by weight (e.g. at least 60%, 70%, 80%, 90%, 95%, 97%, 98%, 99% or more) of the total protein in the composition. The antibodies are thus in purified form.

Pharmaceutical compositions of the invention are preferably supplied in hermetically-sealed containers.

The invention also provides a method of preparing a pharmaceutical composition, comprising a step of admixing an antibody of the invention with one or more pharmaceutically acceptable ingredients.

### Medical Treatments and Uses

Antibodies of the invention may be used for the treatment and/or prevention of disease caused by Neisseria meningitidis, such as meningococcal meningitis, septicaemia or hemorrhagic disease, particularly in humans. Thus the invention provides an antibody of the invention for use in therapy. Also provided is a method of treating a patient comprising administering to that patient an antibody of the invention. Also provided is the use of an antibody of the invention, in the manufacture of a medicament for the treatment and/or prevention of disease caused by N. meningitidis. Also provided is an antibody of the invention for use in the treatment and/or prevention of disease caused by N. meningitidis.

Antibodies can be used for immunoprophylaxis (passive immunization) and/or immunotherapy. To confirm prophylactic efficacy without imposing an infectious challenge on a patient, circulating antibody levels can be tested e.g. in a neutralization assay. To confirm therapeutic efficacy after administration of a pharmaceutical composition of the invention, any known methods for assessing the presence and/or severity of meningococcal infection can be used.

Pharmaceutical compositions of the invention may be administered by any number of routes including, but not limited to, intravenous, intramuscular, intra-arterial, intramedullary, intraperitoneal, intrathecal, intraventricular, transdermal, transcutaneous, oral, topical, subcutaneous, intranasal, enteral, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide some guidance relating to frequency of administration e.g. whether a pharmaceutical should be delivered daily, weekly, monthly, etc. Frequency and dosage may also depend on the severity of symptoms.

Patients will receive an effective amount of the active ingredient i.e. an amount that is sufficient to treat, ameliorate, or prevent meningococcal disease and/or infection. Therapeutic effects may also include reduction in physical symptoms. The optimum effective amount for any particular subject will depend upon their size and health, the nature and extent of the condition, and the therapeutics or combination of therapeutics selected for administration. The effective amount delivered for a given situation can be determined by routine experimentation and is within the judgment of a clinician. For purposes of the present invention, an effective dose will generally be from about 0.01 mg/kg to about 50 mg/kg, or about 0.05 mg/kg to about 10 mg/kg of the compositions of the present invention in the individual to which it is administered. Known antibody-based pharmaceuticals provide guidance in this respect e.g. Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m²; Synagis™ is administered intramuscularly at 15 mg/kg body weight, typically once a month during the RSV season; etc.

Antibodies of the invention may be administered (either combined or separately) with other therapeutics e.g. with an antibiotic effective against meningococcus.

Antibodies of the invention may be useful in treating a hemorrhagic syndrome [60]. Patients treated according to the invention ideally have a functional C3 component.

fHbp

Preferred antibodies of the invention can bind to fHbp from N. meningitidis strain MC58. They will usually be able to bind to fHbp from other meningococcal strains, as well as to artificial or mutant forms of fHbp. For instance, the antibodies may bind to other fHbp sequences within fHbp family I. They may bind to fHbp sequences within fHbp families II and/or III.

Antibodies may bind to an epitope of MC58 fHbp that includes amino acids Gly-148, Arg-149 and Arg-204. Antibodies may bind to an epitope of MC58 fHbp that includes amino acids Pro-145, Gly-147, Gly-148, Arg-204.

Additional amino acids that may be comprised in epitopes bound by antibodies of the invention include, but are not limited to:
Phe-141
Lys-143
Glu-146
Ala-174
Lys-199
Lys-203
Ala-206
Val-207
Phe-227
Gly-228
Lys-230
Glu-233

One way of indicating if a particular residue is present within an antibody's fHbp epitope is to test binding of the antibody to the same fHbp but with that particular residue mutated. Loss of antibody binding indicates that the epitope includes that residue, although it is also helpful to check that the protein has correctly folded etc. to rule out other possibilities.

Another way of indicating if a particular residue is present within an antibody's fHbp epitope is to analyse the fHbp by NMR in the presence and absence of the antibody. If suitable NMR conditions are used, such as a correlation spectroscopy experiment, amino acid residues in the antibody that contact residues in the fHbp can be detected.

Preferred antibodies are bactericidal i.e. they can induce complement-mediated killing of meningococcal bacteria (e.g. strain MC58). Details of a suitable assay are given in reference 13.

In some embodiments of the invention, the antibody is not MAb502 and does not have (i) a heavy chain comprising amino acid sequence SEQ ID NO: 2 and/or (ii) a light chain comprising amino acid sequence SEQ ID NO: 3.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Different steps in a method of the invention can optionally be performed at different times by different people in different places (e.g. in different countries).

### MODES FOR CARRYING OUT THE INVENTION

### Antibody Sequencing

MAb502 is an IgG2a-isotype anti-fHbp monoclonal antibody purified from mouse ascites fluid that is bactericidal against strain MC58.

The heavy chain sequence of the Fv region of MAb502 was found to be SEQ ID NO: 2:

MMVLSLLYLLTALPGILSEVQLQESGPGLAKPSQTLSLTCSVTGYSITSD
FWNWIRKFPGNKLEYMGYISYSGSTDYNPSLKSRISITRDTSKNQYYLQL
NSVTAEDTATYYCARYFGSSYAMDHWGQGTSVTVSSAKTTAPPVYPLVPG
SL

The light chain sequence of the Fv region of MAb502 was found to be SEQ ID NO: 3:

MSVLTQVLGLLLLWLTGARCDIQMTQSPASLSASVGETVTITCRASGNIH
NYLAWYQQKQGKSPQLLVYTAKTLAEGVPSRFSGSGSGTQFSLKINSLQP
EDFGSYYCQHFWSTPWTFGGGTKLEIKRADAAPTVSIFPPSSKLG

Hypervariable loops within SEQ ID NOs: 2 and 3 are underlined and as follows:

| | CDR | | | | | |
|---|---|---|---|---|---|---|
| | H1 | H2 | H3 | L1 | L2 | L3 |
| Residues | 44-53 | 68-76 | 116-125 | 44-54 | 70-76 | 109-117 |
| SEQ ID NO: | 4 | 5 | 6 | 7 | 8 | 9 |

### Epitope Mapping

To test the binding of MAb502 to fHbp produced by natural isolates representative of the diverse meningococcal population we selected 10 strains, including MC58, each of them carrying non-redundant fHbp sequences. Anti-MC58 fHbp polyclonal serum recognized the protein in western blot on the cell extracts of all the strains tested and FACS experiments confirmed the presence of the protein on the bacterial cell surface. As expected, the polyclonal serum elicited by the recombinant protein of MC58 was also able to induce complement-mediated killing of all strains. MAb502 was able to recognize fHbp in 6 out of 10 strains in western blot and FACS experiments and had a strong bactericidal against strain MC58.

Residues involved in the formation of the MAb502 epitope were indicated by analyzing the sequence differences between the fHbp protein produced by MC58 strain and those of the other clinical isolates. Sequence alignment allowed classifying in two groups the amino acid positions variable between fHbpC of MC58 and the other alleles. The first one contained amino acids varying between MC58 and both Western blot-positive and -negative strains. These amino acids corresponded in MC58 to Phe109, Ile114, His119, Glu146, Arg149, Gly163, Ala174, Asn178, Asp192, Ala195, Ala196, Asp197, Gly202, Ala217, Lys230, Lys241, Val243 and Arg247. As the variability of these residues did not impact the western blotting recognition by MAb502, we classified their relative positions as dispensable.

The second group contained residues varying wrt MC58 only in WB-negative strains. These amino acids are Ser117, Gly121, Gln128, Arg130, Pro145, Gly147, Gly148, Thr167 and Arg204. Among these, Ser117, Gly121 & Gln128 are located in the N-terminal flexible tail. With the exception of Thr167, all other residues of this second group cluster on the same region on the β-barrel.

The substitution Arg204→His was the sole difference observed consistently in all the WB-negative strains. The relevance of this position for MAb502 binding was confirmed by site-directed mutagenesis on the recombinant fHbp from MC58. When the Arg204 was changed to histidine, Mab502 was unable to recognize the protein. In close proximity to Arg204 were located three amino acids variable only in WB-negative strains, namely Pro145, Gly147 and Gly148. On the basis of this observation, the key residues of the epitope recognized by MAb502 seem to be Arg204 together with Pro145, Gly147 and Gly148.

We speculated that an additional contribution to the fHbp affinity for MAb502 could be given by the side chains of Glu146, Arg149, Ala174 and Lys230, located in proximity of the residues predicted as crucial for epitope formation.

In order to verify the assumption that certain other residues have minor or no effect on Mab502 binding, we tested by WB the effects of the substitutions Gly202→Lys and Asn178→His. Both mutations, as expected, did not impair the protein recognition by MAb502 in WB.

The predictions on fHbp residues interacting with MAb502 were tested through NMR spectroscopy, by analysing the perturbations caused in the ¹H-¹⁵N HSQC spectrum of fHbpC upon addition of a FAb fragment based on MAb502 (FAb502). Addition of FAb502 to ¹⁵N-fHbpC caused the progressive reduction in intensity of NH cross-peaks in the spectra, which completely disappeared at the 1:1 ratio. The only exception was represented by residues 100-136 belonging to the N-terminal tail, which maintain their full intensity. Disappearance of the protein backbone NHs in the HSQC spectra is a consequence of the formation of the antibody-protein complex, characterized by a high molecular weight (˜70 kDa). The progressive disappearance of fHbpC signals upon addition of FAb502 and the absence of any signal shifting suggests that the complex exchanges with the free protein at rates slower than the chemical shift differences between the two forms, i.e. in the range of milliseconds. The high mobile N-terminal tail, which reorients faster than the overall tumbling rate of fHbpC, maintained this property in the complex. This behaviour indicated that it was not involved in the interaction with FAb2502. Similar chemical shift values for the N-terminal region in both fHbpC and fHbpC-FAb502 complex also indicated that no significant conformational changes occurred in this region. When CRINEPT NMR experiments, which are suitable for high molecular weight systems, were performed, the majority of the NH cross peaks reappeared. The residues of fHbpC, which experienced different chemical shift upon addition of FAb502, are as follows: His138, Thr139, Ser140, Phe141, Lys143, Leu144, Gly148, Arg149, Ala150, Leu166, Ala174, Lys175, Gln176, Gly177, Asn178, Gly179, Ala196, Ile198, Lys199, Lys203, Arg204, His205, Ala206, Val207, Gly210, Ser211, Val212, Leu224, Gly225, Ile226, Phe227, Gly228, Glu233 and Ala238. These residues cluster in one region of fHbpC and include Arg204, the residue essential for binding to the antibody, as well as Gly148, Arg149 and Ala174. In addition, spectral perturbations were observed also for NH cross peaks of other the surface-exposed residues, namely Phe141, Lys143, Lys199, Lys203, Ala206, Val207, Phe227, Gly228 and Glu233.

Further support came from computer modelling of the structure of the complex between fHbpC and the hypervariable antigen-binding fragment of MAb502 (Fv502). Two different models were obtained. In one, the list of fHbp residues contacting Fv502 was: Phe141, Lys143, Pro145, Glu146, Gly147, Gly148, Arg149, Arg204, Phe227, Gly228, Lys230, Glu233; in the other model the list was: Phe141, Lys143, Glu146, Lys199, Lys203, Arg204, Phe227, Gly228, Lys230, Glu233. The two lists have some common features, such as the involvement of Arg204. Another region of interaction present in both sets involves residues Phe227-Lys230, located in the loop comprised between beta strands 6 and 7 of fHbpC.

It will be understood that the invention has been described by way of example only and modifications may be made whilst remaining within the scope and spirit of the invention.

### REFERENCES

[1] Jodar et al. (2002) Lancet 359(9316):1499-1508.
[2] Pizza et al. (2000) Science 287:1816-1820.
[3] WO99/57280.
[4] Masignani et al. (2003) J Exp Med 197:789-799.
[5] Welsch et al. (2004) J Immunol 172:5605-15.
[6] Hou et al. (2005) J Infect Dis 192(4):580-90.
[7] WO03/063766.
[8] Fletcher et al. (2004) Infect Immun 72:2088-2100.
[9] Zhu et al. (2005) Infect Immun 73(10):6838-45.
[10] Cantini et al. (2006) J. Biol. Chem. 281:7220-7227
[11] Welsch et al. (2004) J Immunol 172:5606-15.
[12] Beernink et al. (2007) J Infect Dis. 195(10): 1472-1479.
[13] Giuliani et al. (2005) Infect. Immun. 73, 1151-1160
[14] WO2004/076677.
[15] Traggiai et al. (2004) Nat Med. 10(8):871-5.
[16] Green (1999) J Immunol Methods. 231(1-2):11-23.
[17] Mancini et al. (2004) New Microbiol. 27(4):315-28.
[18] Ewert et al. (2004) Methods 34(2):184-99.
[19] Riechmann et al. (1988) Nature 332:323-327.
[20] O'Brien & Jones (2003) Methods Mol Biol. 207:81-100.
[21] Iwahashi et al. (1999) Mol Immunol. 36(15-16):1079-91.
[22] Lo (2004) Methods Mol Biol. 248:135-59.
[23] Verhoeyen et al. (1988) Science 239: 1534-1536.
[24] Kabat et al. (1991) Sequences of Proteins of Immunological Interest, 5th Ed. Public Health Service, National Institutes of Health, Bethesda, Md.
[25] Chothia & Lesk (1987) Mol. Biol. 196:901-917.
[26] Kashmiri et al. (2005) Methods 36(1):25-34.
[27] Gonzales et al. (2004) Mol Immunol. 41(9):863-72.
[28] Worn & Pluckthun (2001) J Mol Biol. 305(5):989-1010.
[29] WO93/16185
[30] Adams & Schier (1999) J Immunol Methods. 231(1-2):249-60.
[31] Hallborn & Carlsson (2002) Biotechniques Suppl:30-7.
[32] Pini & Bracci (2000) Curr Protein Pept Sci 1(2):155-69.
[33] Walter et al. (2001) Comb Chem High Throughput Screen. 4(2):193-205.
[34] Gruber et al. (1994) J Immunol 152(11):5368-74.
[35] U.S. Pat. No. 5,591,828
[36] WO 93/11161.
[37] Hollinger et al. (1993) Proc. Natl. Acad. Sci. USA 90:6444-6448.
[38] Hudson & Kortt (1999) J Immunol Methods 231:177-89.
[39] Muyldermans (2001) J Biotechnol 74(4):277-302.
[40] Dumoulin et al. (2002) Protein Sci. 11(3):500-15.
[41] Sidhu et al. (2004) J Mol Biol. 338(2):299-310.
[42] Kotz et al. (2004) Eur J. Biochem. 271(9):1623-9.
[43] Herrin et al. (2008) Proc. Natl. Acad. Sci. USA 105:2040-5.
[44] WO2008/016854.
[45] U.S. Pat. No. 5,624,821.
[46] U.S. Pat. No. 6,737,056.
[47] U.S. Pat. No. 6,538,124.
[48] U.S. Pat. No. 6,528,624.
[49] Shields et al. (2001) J Biol Chem 276:6591-604.
[50] Idusogie et al. (2000) J Immunol 164(8):4178-84.
[51] Dall'acqua et al. (2006) J Biol Chem 281(33):23514-24.
[52] Marks et al. (1992) Bio/Technology 10:779-83.
[53] Wu et al. (2005) J Mol Biol 350(1):126-44.
[54] Chapter 4 of Kuby Immunology (4th edition, 2000; ASIN: 0716733315
[55] Jones et al. Biotechnol Prog 2003,19(1):163-8
[56] Cho et al. Cytotechnology 2001,37:23-30
[57] Cho et al. Biotechnol Prog 2003,19:229-32
[58] Gennaro (2000) Remington: The Science and Practice of Pharmacy. 20th edition, ISBN: 0683306472.
[59] U.S. Pat. No. 7,132,100.
[60] WO2008/125985.

1. An antibody having (i) a heavy chain comprising amino acid sequence SEQ ID NO: 2 and/or (ii) a light chain comprising amino acid sequence SEQ ID NO: 3.

2. An antibody that binds to an epitope of MC58 fHbp, wherein the epitope comprises amino acids Gly-148, Arg-149 and Arg-204.

3. An antibody that binds to an epitope of MC58 fHbp, wherein the epitope comprises amino acids Pro-145, Gly-147, Gly-148 and Arg-204.

4. An antibody having one or more CDRs from within SEQ ID NO: 2.

5. An antibody having one or more CDRs from within SEQ ID NO: 3.

6. An antibody that binds to the same epitope as the antibody of claim 1.

7. An antibody that competes for binding to fHbp with the antibody of claim 1.


SEQUENCE LISTING (9 sequences)

SEQ ID NO: 1 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 152 aa, Mus musculus
MMVLSLLYLLTALPGILSEVQLQESGPGLAKPSQTLSLTCSVTGYSITSDFWNWIRKFPGNKLEYMGYISYSGSTDYNPSLKSRISITRDTSKNQYYLQLNSVTAEDTATYYCARYFGSSYAMDHWGQGTSVTVSSAKTTAPPVYPLVPGSL

SEQ ID NO: 3 - protein, 145 aa, Mus musculus
MSVLTQVLGLLLLWLTGARCDIQMTQSPASLSASVGETVTITCRASGNIHNYLAWYQQKQGKSPQLLVYTAKTLAEGVPSRFSGSGSGTQFSLKINSLQPEDFGSYYCQHFWSTPWTFGGGTKLEIKRADAAPTVSIFPPSSKLG

SEQ ID NO: 4 - protein, 10 aa, Mus musculus
GYSITSDFWN

SEQ ID NO: 5 - protein, 9 aa, Mus musculus
YISYSGSTD

SEQ ID NO: 6 - protein, 10 aa, Mus musculus
YFGSSYAMDH

SEQ ID NO: 7 - protein, 11 aa, Mus musculus
RASGNIHNYLA

SEQ ID NO: 8 - protein, 7 aa, Mus musculus
TAKTLAE

SEQ ID NO: 9 - protein, 9 aa, Mus musculus
QHFWSTPWT